(19) United States
(12) Patent Application Publication
(10) Pub. No.: US 2026/0248483 A1
(43) Pub. Date: Aug. 27, 2026

(54) System and Method for a Vessel Assessment Tool

(71) Applicant: Bard Access Systems, Inc., Salt Lake City, UT (US)

(72) Inventors: Bart D. Peterson, Farmington, UT (US); Bradley M. Wilkinson, North Haledon, NJ (US); Kelly E. Harper, West Valley City, UT (US); Tab Robbins, Layton, UT (US); Matthew J. Prince, Herriman, UT (US)

(21) Appl. No.: 19/652,900

(22) Filed: Apr. 20, 2026

Related U.S. Application Data
(63) Continuation of application No. 17/707,662, filed on Mar. 29, 2022, now Pat. No. 12,605,140.
(60) Provisional application No. 63/167,219, filed on Mar. 29, 2021.

(51) Int. Cl. A61B 8/08; A61B 8/00

(52) U.S. Cl. CPC A61B 8/0891; A61B 8/463; A61B 8/465

(57) ABSTRACT

A vessel assessment system includes an ultrasound probe and a console coupled to the ultrasound probe, the console comprising a display and one or more logic modules coupled to one or both of a processor and a data store. The one or more logic modules can include an imaging logic, a vessel selection logic, and a vascular access device (VAD) selection logic. The imaging logic can be configured to provide an ultrasound image from the ultrasound probe on the display, the ultrasound image including one or more blood vessels. The vessel selection logic can be configured to superimpose and align one or more vessel selection rings on the ultrasound image. The VAD selection logic can be configured to automatically select the one or more vessel selection rings, each representing a catheter gauge that can successfully access a blood vessel of the one or more blood vessels on the ultrasound image.

### PRIORITY

This application is a continuation of U.S. Patent Application No. 17/707,662, filed March 29, 2022, now U.S. Patent No. 12,605,140, which claims the benefit of priority to U.S. Provisional Application No. 63/167,219, filed March 29, 2029, each of which is incorporated by reference in its entirety into this application new paragraph.

### SUMMARY

Embodiments disclosed herein are directed to a system and method for a vessel assessment tool configured to image a target vessel, disposed subcutaneously, and determine which vascular access device to use to access the target vessel, while remaining within one or more predetermined parameters of percentage vessel occupancy, and dwell length given a predetermined range of insertion angles.

When placing a vascular access device (“VAD”) such as a catheter, central venous catheter (“CVC”), peripherally inserted central catheter (“PICC”), peripheral intravenous catheter (“PIV”), or the like, clinicians must select a VAD based upon a number of different parameters. Current systems require clinicians to select a particular VAD first and then use various imaging tools to evaluate if the selected VAD is appropriate or not. This necessitates a certain amount of “trial and error” on the part of the clinician to select the correct VAD prior to locating an appropriate vessel to access.

Embodiments described herein are directed to a vessel assessment tool configured to image one or more target vessels of a patient and, according to one or more predetermined parameters, provide one or more VAD tools that are most suited to accessing the vessel.

Disclosed herein is a vessel assessment system including, an ultrasound probe, and a console communicatively coupled to the ultrasound probe, the console including a display and one or more logic modules coupled to one or both of a processor and a data store, the one or more logic modules including, an imaging logic configured to provide an image of a target vessel, a vessel selection logic configured to determine one or both of a diameter and a cross-sectional area of the target vessel, and a VAD selection logic configured to retrieve an insertion parameter, and select a VAD icon from a list of VAD icons, the VAD icon representing a vascular access device configured to access the target vessel within the insertion parameter.

In some embodiments, the ultrasound probe is configured to send an acoustic signal and detect a reflected acoustic signal, and the imaging logic is configured to retrieve information from the ultrasound probe and provide an image of the target vessel disposed subcutaneously.

In some embodiments, the vessel selection logic is configured to retrieve image information from the imaging logic, and automatically align a vessel selection ring, superimposed on the image, with the target vessel.

In some embodiments, the vessel selection logic is configured to receive an input from a user to manipulate a vessel selection ring, superimposed on the image, to align with the target vessel.

In some embodiments, the vessel selection ring can change one or more of a diameter and a shape to align the vessel selection ring with the target vessel.

In some embodiments, the insertion parameter includes one or more of a percentage vessel occupancy parameter, a dwell length parameter, and an insertion angle parameter.

In some embodiments, the VAD selection logic is further configured to superimpose the VAD icon on the image, and align the VAD icon with the target vessel.

In some embodiments, the size of the VAD icon is scaled relative to the target vessel to provide a visual comparison of the vascular access device relative to the target vessel.

In some embodiments, the VAD icon of the list of VAD icons includes a specification measurement of the vascular access device, each VAD icon of the list of one or more VAD icons representing a different vascular access device.

Also disclosed is a vessel assessment tool including, a processor, and memory communicatively coupled to the processor, the memory including machine readable instructions that when executed by the processor, cause the processor to execute, i) an imaging logic configured to provide an image of a target vessel disposed subcutaneously, ii) a vessel selection logic configured to determine one or more of a diameter, a cross-sectional area, and a depth of the target vessel, and iii) a VAD selection logic configured to retrieve information from the vessel selection logic, and retrieve an insertion parameter, and provide a VAD icon representing a vascular access device configured to access the target vessel within the insertion parameter.

In some embodiments, the vessel assessment tool further includes an ultrasound probe configured to send and receive an acoustic signal, and wherein the imaging logic is configured to retrieve information from the ultrasound probe to provide the image of the target vessel.

In some embodiments, the vessel selection logic is further configured to identify one or more features of the image, and automatically align a vessel selection ring, superimposed on the image, with the target vessel.

In some embodiments, the vessel selection logic is further configured to receive an input from a user to manipulate a vessel selection ring, superimposed on the image, to align with the target vessel.

In some embodiments, the vessel selection logic is configured to change one or more of a position, a diameter, an area, and a shape of the vessel selection ring, to align the vessel selection ring with the target vessel.

In some embodiments, the VAD icon is superimposed on the image, and aligned with the target vessel.

In some embodiments, the size of the VAD icon is scaled relative to the image to provide a visual comparison of the vascular access device relative to the target vessel.

In some embodiments, the VAD selection logic provides a list of one or more VAD icons, each VAD icon of the list of one or more VAD icons representing a different vascular access device.

In some embodiments, the VAD selection logic is configured to receive an input to select a second VAD icon from the list of one or more VAD icons, representing a second vascular access device.

Also disclosed is a method of selecting a vascular access device for accessing a target vessel including, detecting a reflected ultrasonic acoustic signal using an ultrasonic imaging device, providing an image of the target vessel disposed subcutaneously, determining one or more of a depth, a diameter, and a cross-sectional area of the target vessel, retrieving an insertion parameter including one or more of a maximum percentage vessel occupancy parameter, a minimum dwell length parameter, and an insertion angle parameter, and automatically selecting, by a VAD selection logic, a vascular access device, a diameter of the vascular access device being equal to or less than the maximum percentage vessel occupancy parameter, or a dwell length metric of the vascular access device being equal to or greater than the minimum dwell length parameter.

In some embodiments, the method further includes the VAD selection logic automatically aligning a vessel selection ring, superimposed on the image, with the target vessel.

In some embodiments, the method further includes providing an input to the VAD selection logic to modify a vessel selection ring, superimposed on the image, to align the vessel selection ring with the target vessel.

In some embodiments, the method further includes retrieving the insertion angle parameter, the depth of the target vessel and a total length of the vascular access device and determining the dwell length metric of the vascular access device.

In some embodiments, the method further includes superimposing a VAD icon on the image of the target vessel, the VAD icon representing one or both of a diameter and a cross-sectional area of the vascular access device relative to the target vessel.

In some embodiments, the method further includes selecting, by a user interface, a second VAD icon representing a second vascular access device, a diameter of the second vascular access device being less than the maximum percentage vessel occupancy parameter, or a dwell length metric of the second vascular access device being greater than the minimum dwell length parameter.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A illustrates an exemplary system configured for targeting a vessel and determining a vascular access device to access the target vessel, in accordance with embodiments disclosed herein.

FIG. 1B illustrates a schematic view of the system of FIG. 1A, in accordance with embodiments disclosed herein.

FIG. 2A illustrates a screenshot of the system of FIG. 1A, in accordance with embodiments disclosed herein.

FIG. 2B illustrates a cross-sectional side view of a vascular access device accessing a target vessel, in accordance with embodiments disclosed herein.

FIG. 3 illustrates a screenshot of a parameter entry screen of the system of FIG. 1A, in accordance with embodiments disclosed herein.

FIG. 4 illustrates a flow chart of a method of use for the system of FIG. 1A, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

### Terminology

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

In the following description, numerous specific details are set forth in order to provide a thorough understanding of the present disclosure. It will be apparent, however, to one of ordinary skill in the art that the invention disclosed herein may be practiced without these specific details. In other instances, specific numeric references such as “a first actionable element,” may be made. However, the specific numeric reference should not be interpreted as a literal sequential order but rather interpreted that the “first actionable element” is different than a “second actionable element.” Thus, the specific details set forth are merely exemplary. The specific details may be varied from and still be contemplated to be within the spirit and scope of the present disclosure. The term “coupled” is defined as meaning connected either directly to the component or indirectly to the component through another component, where the connection may enable communications between these components.

As used herein, the term “communication” generally refers to related data that is received, transmitted, or exchanged within a communication session. The data may include a plurality of packets, where a “packet” broadly refers to a series of bits or bytes having a prescribed format. Alternatively, the data may include a collection of data that may take the form of an individual or a number of packets carrying related payloads, e.g., a single webpage received over a network. Further, as used herein, the terms “about,” “approximately,” or “substantially” for any numerical values or ranges indicate a suitable dimensional tolerance that allows the part or collection of components to function for its intended purpose as described herein.

In the following description, certain terminology is used to describe features of the invention. For example, in certain situations, the term “logic” is representative of hardware, firmware and/or software that is configured to perform one or more functions. As hardware, logic may include circuitry having data processing or storage functionality. Examples of such circuitry may include, but are not limited or restricted to a microprocessor, one or more processor cores, a programmable gate array, a microcontroller, a controller, an application specific integrated circuit (“ASIC”), wireless receiver, transmitter and/or transceiver circuitry, semiconductor memory, or combinatorial logic.

Alternatively, logic may be software, such as executable code in the form of an executable application, an Application Programming Interface (API), a subroutine, a function, a procedure, an applet, a servlet, a routine, source code, object code, a shared library/dynamic load library, or one or more instructions. The software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; semiconductor memory; non-persistent storage such as volatile memory (e.g., any type of random access memory “RAM”); or persistent storage such as non-volatile memory (e.g., read-only memory “ROM,” power-backed RAM, flash memory, phase-change memory, etc.), a solid-state drive, hard disk drive, an optical disc drive, or a portable memory device. As firmware, the executable code may be stored in persistent storage. In an embodiment, the logic described herein may rely on heuristics, machine learning, artificial intelligence (A.I.), neural networks, or other data processing techniques to perform the described functionality.

The term “computing device” may be construed as electronics with data processing capabilities and/or a network interface capabilities, such as network connectivity to a physical or virtual network such as a public network (e.g., Internet), a private network (e.g., a wireless data telecommunication network, a local area network “LAN”, etc.), a public cloud network, a virtual private cloud, of the like. Examples of a computing device may include, but are not limited or restricted to, the following: a server, an endpoint device (e.g., a laptop, a smartphone, a “wearable” device, a smartwatch, a tablet, a desktop or laptop computer, a netbook, or any general-purpose or special-purpose, user-controlled electronic device); a mainframe; a router; or the like.

The term “network” may include a public and/or private network based on wired or wireless interconnects and in a centralized or decentralized configuration. The networks may include, but are not limited or restricted to a Local Area Network (LAN), a Wireless Local Area Network (WLAN), a Virtual Private Network (VPN), intranet, internet, ‘cloud’ based network, or similar network configurations.

A “message” generally refers to information transmitted in one or more electrical signals that collectively represent electrically stored data in a prescribed format. Each message may be in the form of one or more packets, frames, HTTP-based transmissions, or any other series of bits having the prescribed format.

The term “computerized” generally represents that any corresponding operations are conducted by hardware in combination with software and/or firmware.

As used herein, the term “icon” or “VAD icon” can include any alphanumerical or pictorial symbol to represent a device, e.g. a vascular access device.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition may occur only when a combination of elements, functions, steps or acts are in some way inherently mutually exclusive.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

### Vessel Assessment Tool

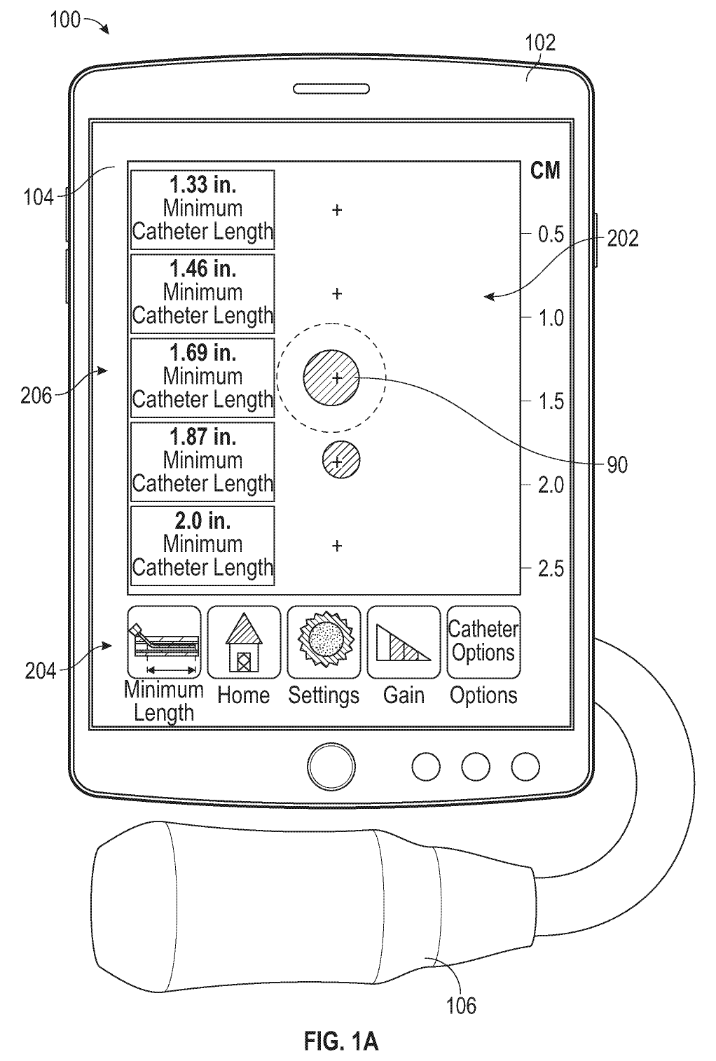

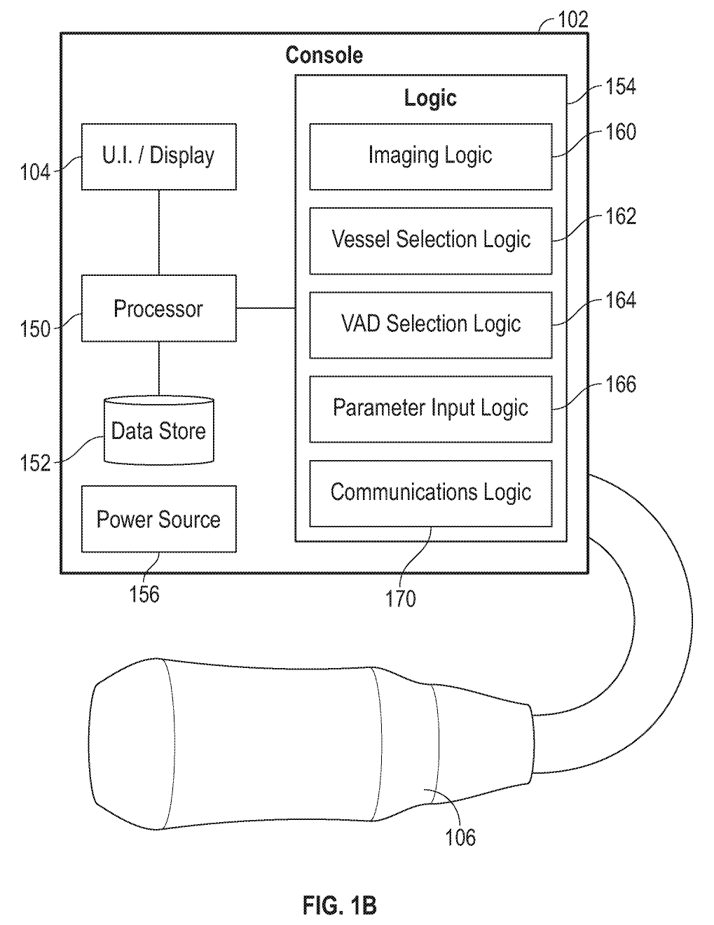

FIG. 1A illustrates an exemplary vessel assessment system (“system”) 100 configured to image a subcutaneous target vessel and determine which vascular access device to use to access the target vessel, while remaining within one or more predetermined insertion parameters. Exemplary insertion parameters can include a percentage vessel occupancy, a dwell length, and an insertion angle or insertion angle range. In an embodiment, a vascular access device (“VAD”) can include but not limited to, a catheter, dialysis catheter, central venous catheter (“CVC”), peripherally inserted central catheter (“PICC”), peripheral intravenous catheter (“PIV”), midline catheter, sacrificial catheter, introducer, trocar, or the like.

The system 100 can generally include a console 102 including a display 104, and a probe 106 communicatively coupled, either wired or wirelessly, to the console 102. In an embodiment, the system 100 can be configured to image a subcutaneous portion of a patient using one or more modalities. Exemplary modalities can include, but not limited to, electromagnetic, permanent (static) magnetic, optical, acoustic modalities, combinations thereof, or the like. In an embodiment, the system 100 can be configured to use an ultrasound modality to image a target vessel 90 within the patient. In an embodiment, the probe 106 can include a medical device guide (not shown) configured to guide an insertion angle for a VAD within a range of insertion angles.

In an embodiment, the console 100 can include one or more user interface elements 104, such as buttons, keyboards, “mouse”, trackpad, trackball, touch screens, voice recognition inputs, combinations thereof, or the like. In an embodiment, the display 104 may be a touch screen and can be included with the console 102. In an embodiment, the display 104 can be a separate stand-alone unit from the console 102 and can be communicatively coupled, either wired or wirelessly, with the console 102. Exemplary wireless communication modalities can include WiFi, Bluetooth, Near Field Communications (NFC), cellular Global System for Mobile Communication (“GSM”), electromagnetic (EM), radio frequency (RF), combinations thereof, or the like.

FIG. 1B shows a schematic view of the vessel assessment system 100 of FIG. 1A, the console 102 can include one or more processors 150, data store 152, and one or more logic engines 154, such as an imaging logic 160, a vessel selection logic 162, a VAD selection logic 164, a parameter input logic 166, and a communications logic 170, as described in more detail herein. In an embodiment, the system 100 can further include an internal power source 156 or be powered by a mains power supply.

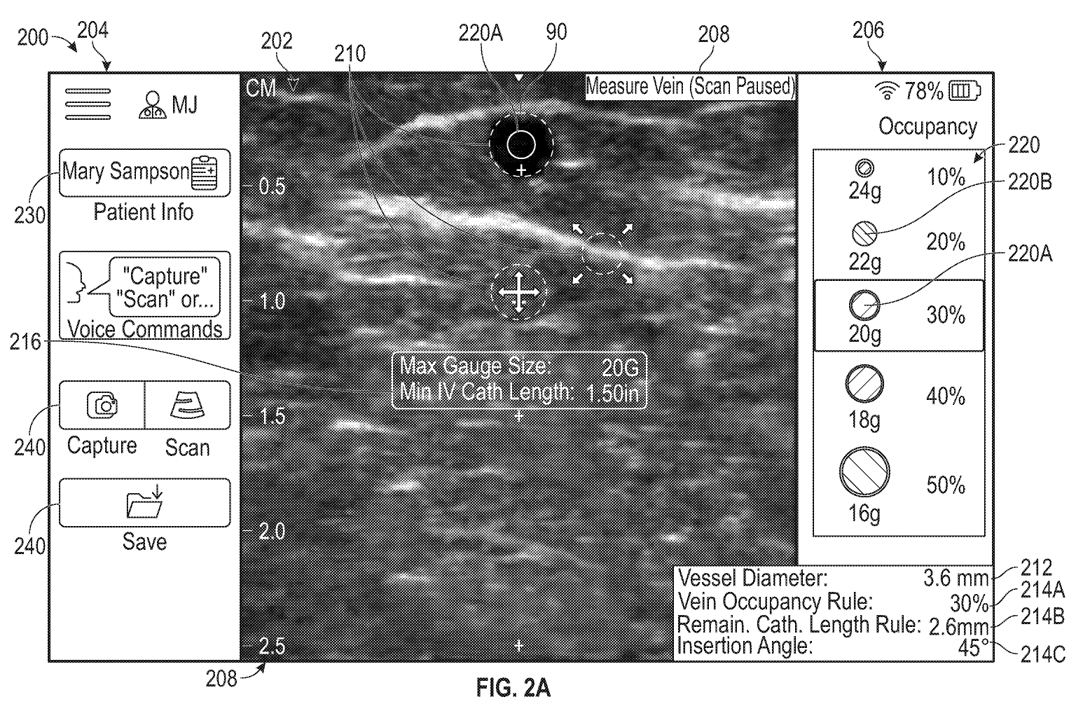

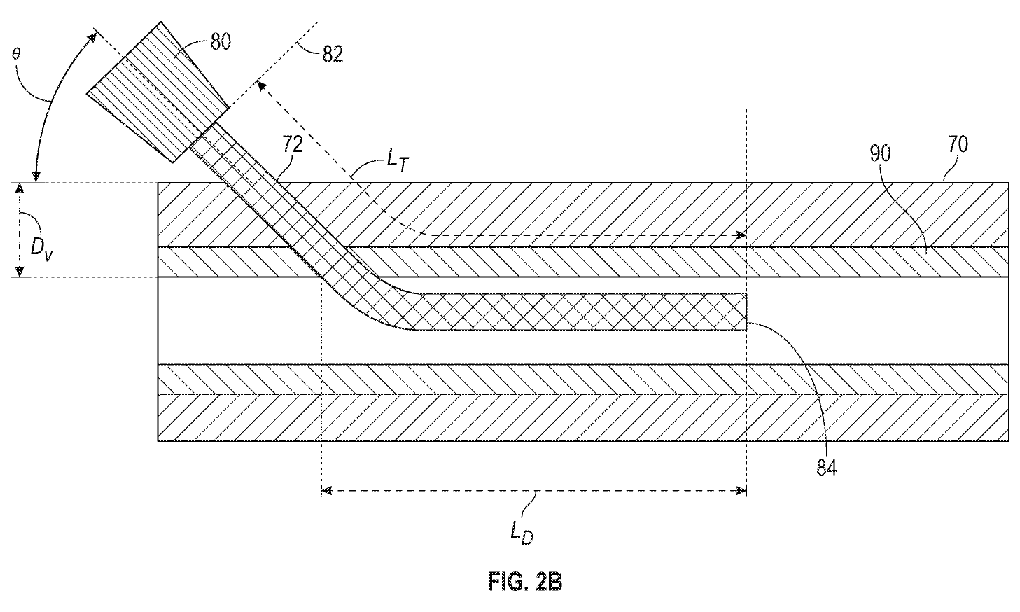

FIG. 2A shows an exemplary screenshot 200 from the display 104 of the system 100. In an embodiment, as shown in FIG. 2A, the screenshot 200 can include an image 202 of a subcutaneous portion of the patient (e.g. an ultrasound image) including a target vessel 90, a user interface section 204, and a VAD selection tool 206.

In an embodiment, the user interface section 204 can display various information about the patient such as name, date of birth, patient ID number, and the like, and/or can include a selection button 230 to display further information about the patient. In an embodiment, the system 100 can provide a patient information interface to allow the clinician to enter or update information about the patient by way of the system 100. In an embodiment, information about the patient can be stored locally, e.g. on data store 152 or can be retrieved from a remote database or network by way of the communications logic 170. Exemplary remote databases or networks can include the internet, hospital intranet, electronic health record (EHR) database, combinations thereof, or the like. In an embodiment, the user interface section 204 can further include one or more image control buttons 240 for manipulating the image 202, e.g. freezing or capturing the image, saving the image to the data store 152, etc. In an embodiment, the system 100 can be controlled by voice recognition commands, the voice recognition commands can be displayed on the user interface section 204.

In an embodiment, the imaging logic 160 can be configured to send and receive signals to/from the probe 106 and the display 104, to provide the image 202 of a subcutaneous portion of the patient. In an embodiment, the imaging logic 160 can be an ultrasound imaging logic configured to send and receive signals to/from an ultrasound probe 106. The ultrasound probe 106 can be configured to emit an ultrasonic acoustic signal and detect a reflected ultrasonic acoustic signal. The ultrasound imaging logic 160 can be configured to display an image of the subcutaneous portion of the patient based on the reflected ultrasonic acoustic signal and can be used to locate a target vessel 90. In an embodiment, the imaging logic 160 can provide image information 208 such as a subcutaneous depth scale, current actions, image scanning or pausing, combinations thereof, or the like. In an embodiment, the imaging logic 160 can provide the image control buttons 240 on the display 104.

In an embodiment, a vessel selection logic 162 can be configured to provide a vessel selection ring 210 superimposed on the image 202. The vessel selection logic 162 can be configured to receive an input from the user to move the vessel selection ring 210 relative to the image 202. In an embodiment, the display 104 is a touch screen display and the user can manipulate the vessel selection ring 210 by touching and dragging across the display 104. However, it will be appreciated that the vessel selection logic 162 can be configured to receive other input(s) from the user to move the vessel selection ring 210 such as keyboards, trackpads, track balls or motion detection devices, voice recognition, combinations thereof, or the like.

In an embodiment, the vessel selection logic 162 can be configured to resize the vessel selection ring 210 to align with one or more of a diameter, size and shape of the target vessel 90. In an embodiment, the vessel selection logic 162 can be configured to change the shape of the vessel selection ring 210 (e.g. circular, elliptical, etc.) to align with the shape of the target vessel 90 and to determine one or more of a minimum diameter, maximum diameter, or cross-sectional area of the target vessel 90. In an embodiment, the vessel selection logic 162 can provide one or more vessel selection rings 210 that can each be aligned with one or more target vessels 90. For example, a first vessel selection ring can be aligned with a first target vessel, a second vessel selection ring can be aligned with a second target vessel, etc.

In an embodiment, the vessel selection logic 162 can be configured to recognize one or more features of the image 202 and automatically align the vessel selection ring 210 with the feature of the image. For example, the vessel selection logic 162 can automatically recognize features within the image that indicate a change in tissue structure. These features can indicate a change in acoustic properties of the tissue that can indicate a boundary of one or more different tissue structures or target vessel(s) 90, etc. As such, the vessel selection logic 162 can the automatically identify one or more target vessels 90 within the image 202 and align and resize the vessel selection ring 210 with a target vessel 90 of the one or more target vessels. In an embodiment, the user can select or further manipulate the vessel selection ring 210 after automatic alignment of the vessel selection ring 210 with the target vessel 90. In an embodiment, the vessel selection logic 162 can be configured to display vessel information 212 on the display 104, for one or more target vessels 90. For example, vessel information 212 can include, but not limited to, a vessel diameter (e.g. minimum, maximum, mean, median diameters), a vessel cross-sectional area, a vessel shape, a depth of the vessel below the skin surface, or the like. The vessel information 212 can be dynamically updated based on position or size of the one or more vessel selection rings 210 in relation to the one or more target vessels 90.

In an embodiment, a VAD selection logic 164 can be configured to retrieve information about the target vessel 90 from the vessel selection logic 162, retrieve information about one or more VAD’s, retrieve information about one or more insertion parameters 214, and indicate to the user one or more VAD’s that can successfully access the target vessel 90 while remaining within the predetermined insertion parameters 214. As described herein, information about the target vessel 90 can include a diameter, cross-sectional area, a depth of the target vessel 90 relative to a skin surface, a vessel shape (circular or elliptical), combinations thereof, or the like.

Exemplary information about one or more VAD’s can include, but not limited to, a make, model, part number, serial number, specifications, diameter, gauge (e.g. 24g, 22g, 20g, 18g, 16g, or the like), a VAD length, a VAD dwell length, a preferred insertion angle or insertion angle range, purchase price, current inventory, ordering information, combinations thereof, or the like. The VAD information can be stored locally on the data store 152, or can be communicated to the system 100 by way of the communications logic 170 from a network or remote database (e.g. an electronic health record database, VAD manufacturer’s database, internet, intranet, hospital network, or the like).

As used herein, the insertion parameters 214 can include one or more of a percentage vessel occupancy parameter 214A, a dwell length parameter 214B (either an absolute length metric, or a percentage of total VAD length), and an insertion angle range 214C. However, it will be appreciated that other insertion parameters 214 are also contemplated. The insertion parameters 214 can be entered by a user, derived by the system 100, or can be retrieved from the local data store 152, or retrieved from a network or remote database (e.g. an electronic health record database, VAD manufacturer’s database, etc.).

In an embodiment, a percentage vessel occupancy parameter 214A can be a maximum percentage of the diameter or cross-sectional area of the target vessel 90 that is occupied by the VAD, when the VAD is disposed within the target vessel 90. This can be important since a VAD that is larger than a percentage vessel occupancy parameter can occlude the vessel and mitigate blood flow. As a non-limiting example, where a target vessel 90 has a diameter of 3.65 mm and a vessel occupancy parameter is set to 30%, the vessel occupancy logic 164 can determine that (3.65mm x 30% = 1.1mm) or a 20 gauge (20G) VAD or smaller can be used to access the target vessel 90 while remaining at or below the maximum vessel occupancy parameter.

In an embodiment, a dwell length metric is a length of the VAD that would dwell within target vessel 90, when the VAD accesses the target vessel 90. This can be important since the minimum dwell length metric can ensure that the VAD remains engaged with the vessel throughout the procedure. If the dwell length is too short, then a distal tip of the VAD may be pulled out of the vessel accidentally leading to complications to the patient, aborting the procedure and/or re-accessing the vessel. The dwell length metric can be expressed either as an absolute length, or as a percentage of the total length of the VAD. The dwell length metric can be the length of the VAD disposed within the target vessel 90, given the total length of the VAD. As described in more detail herein, the dwell length of a given VAD can be affected by the depth of the target vessel 90 below the skin surface, and the insertion angle of the VAD.

In an embodiment, the VAD selection logic 164 can be configured to display the VAD selection tool 206 on the display 104. The VAD selection tool 206 can display one or more VAD icons 220 representing one or more VAD’s and can include information associated therewith, such as VAD cross-section diameter or gauge, a VAD total length, a percentage vessel occupancy metric or a dwell length metric relative to the target vessel 90, or the like. In an embodiment, an “icon” can include any alphanumerical or pictorial symbol, shape color or graphical representation (e.g. chart, graph, Venn diagram, radar chart, etc.), to represent a device or range of devices. For example, a first VAD icon can represent a first vascular access device or first group of devices, and can differentiate the first VAD icon from a second VAD icon representing a second vascular access device or group of devices. The VAD icon may include one or more specification measurements (length, diameter, gauge, etc.), serial numbers, make, model number, color or any defining information, combinations thereof, or the like, to facilitate differentiating the associated vascular access device or group of devices from a different vascular access device or group of devices. In an embodiment, the VAD icon 220 can be color coded, or include a distinct pattern or symbol to easily differentiate between the different VAD’s and associated VAD information. The VAD selection logic 164 can be configured to receive an input from the user to select a first VAD icon 220A, and the VAD selection logic 164 can provide a first VAD icon 220A superimposed on the image 202. In an embodiment, the first VAD icon 220A can be aligned with a center of the target vessel selection ring 210.

In an embodiment the size (e.g. diameter) of the VAD icon 220 can be scaled relative to the image and the size of the target vessel 90. As such, when the VAD icon 220A is superimposed on the image 202, the user can immediately see a size of the VAD relative to the target vessel 90. Advantageously, the size of the VAD icon 220A can allow a user to quickly see if the VAD selection is appropriate. In an embodiment, the VAD selection logic 164 can be configured to provide one or more alerts to indicate to the user if the VAD selected, e.g. the first VAD icon 220A, is within one or more of the insertion parameters 214. Exemplary alerts can be audible, visual or tactile alerts.

In an embodiment, a minimum dwell length parameter can be a minimum longitudinal length of the VAD 80 that is disposed within the target vessel 90 when the VAD accesses the target vessel 90. The minimum dwell length parameter can be expressed either as an absolute length (e.g. in mm or cm), or as a percentage of the total VAD length (L T). FIG. 2B shows a longitudinal cross-sectional view of an exemplary VAD 80 disposed within a target vessel 90. The VAD 80 can define a total VAD length (L T) extending from a proximal end 82 to a distal tip 84. A dwell length (L D) can be a length of the VAD 80 that is disposed within the vessel 90. As will be appreciated, for the VAD 80 to remain engaged with the target vessel 90, a length of the VAD 80 must extend through the insertion site 72 and into the vessel 90. As will be appreciated, various factors can affect the dwell length (L D) for a given VAD 80. These factors can include the total VAD length (L T), the depth (D V) of the target vessel 90 relative to the skin surface 70, and the angle of insertion (Ɵ) of the VAD 80 relative to the skin surface 70. In an embodiment, the insertion angle (Ɵ) can be between 1^{o} and 90^{o} relative to the skin surface 70.

In an embodiment, the VAD selection logic 164 can be configured to retrieve information on the depth (D V) of the target vessel 90 from the vessel selection logic 162, the insertion angle (Ɵ) 214C or range of insertion angles (Ɵ) from the insertion parameters 214 and information on the total VAD length (L T) to determine a dwell length (L D), and determine if the dwell length (L D) is less than a minimum dwell length parameter 214B.

In an embodiment, the VAD selection logic 164 can select a VAD icon 220 that represents a VAD 80 that can access the target vessel 90, while remaining within one or more of the insertion parameters 214. In an embodiment, the VAD selection logic 164 can provide a list of one or more VAD icons 220, each VAD icon 220 of the list of VAD icons can represent a VAD 80 of one or more VAD’s that can access the target vessel 90, while remaining within one or more of the insertion parameters 214. In an embodiment, the VAD selection logic 164 can automatically select a VAD icon 220, as described herein, a user can then select a second VAD icon 220B that represents a different VAD with different specifications that can also access the target vessel 90, while remaining within one or more of the insertion parameters 214. Advantageously, the system 100 can automatically select a VAD icon 220 for the user to access the target vessel 90, and provide a VAD with the largest possible gauge (i.e. largest lumen diameter), while remaining within one or more of the insertion parameters 214. Further the user can select a different VAD icon, representing a different VAD device that can also access the target vessel 90, while remaining within one or more of the insertion parameters 214.

With further reference to FIGS. 2A-2B, in an embodiment, the VAD selection logic 164 can provide alerts and/or VAD information 216 directed to the selected VAD icon 220, e.g. the first VAD icon 220A, superimposed on the screen 104, or information about the one or more VAD’s that are able to access the target vessel 90. Advantageously, the system 100 can image a subcutaneous portion of the patient, identify a target vessel 90, determine a cross-sectional area or diameter of the vessel 90 and a depth of the vessel relative to the skin surface 70. The system 100 can then determine which VAD 80 can be used to access the vessel 90 while remaining within one or more insertion parameters 214, e.g. the percentage vessel occupancy parameter 214A, and dwell length parameter 214B, based on the specifications of the VAD and an angle, or range of angles, of insertion 214C. As such, the system 100 can advise the clinician on which VAD(s) 80 to use to access the target vessel 90.

In an exemplary method of use, a system 100 is provided, as described herein. A clinician can image a portion of the patient including the target vessel 90 using the probe 106. In an embodiment, the system 100 uses an ultrasound modality to image the patient, however, it will be appreciated that other modalities are also contemplated. An image 202 of the target vessel 90 can be provided on the display 104 and a clinician can manipulate a target vessel selection ring 210 to position and align with a target vessel 90 in the image 202. Optionally, the system 100 can automatically align a vessel selection ring 210 with a target vessel 90 in the image 202. In an embodiment, one or more vessel selection rings 210 can be aligned with one or more target vessels 90 in the image 202. For example, a first vessel selection ring can be aligned with a first target vessel, a second vessel selection ring can be aligned with a second target vessel, etc.

The system 100 can then determine a cross-sectional size and depth of the target vessel 90 based on the size, shape and position of the vessel selection ring 210 within the image 202. The system 100 can retrieve one or more insertion parameters 214 and retrieve information about one or more VAD’s 80, either from the data store 152 or from a network or remote database, as described herein. Optionally, the clinician can enter the insertion parameters 214 or the information about one or more VAD’s 80 to the system 100 either before or after imaging the target vessel 90. The system 100 can then determine which VAD can access the target vessel 90 while remaining within the insertion parameters 214, as described herein. In an embodiment, the system 100 can automatically select a VAD, represented by a VAD icon 220 that can access the target vessel 90 to provide, for example, the largest gauge VAD or shortest total VAD length (L T), while remaining within the insertion parameters 214. In an embodiment a clinician can select a VAD icon 220 representing a VAD 80. The system 100 can display the icon 220 within the target vessel 90. Further the system 100 can display information to the clinician, including the insertion parameters 214, target vessel information, VAD information, patient information, system information, and the like.

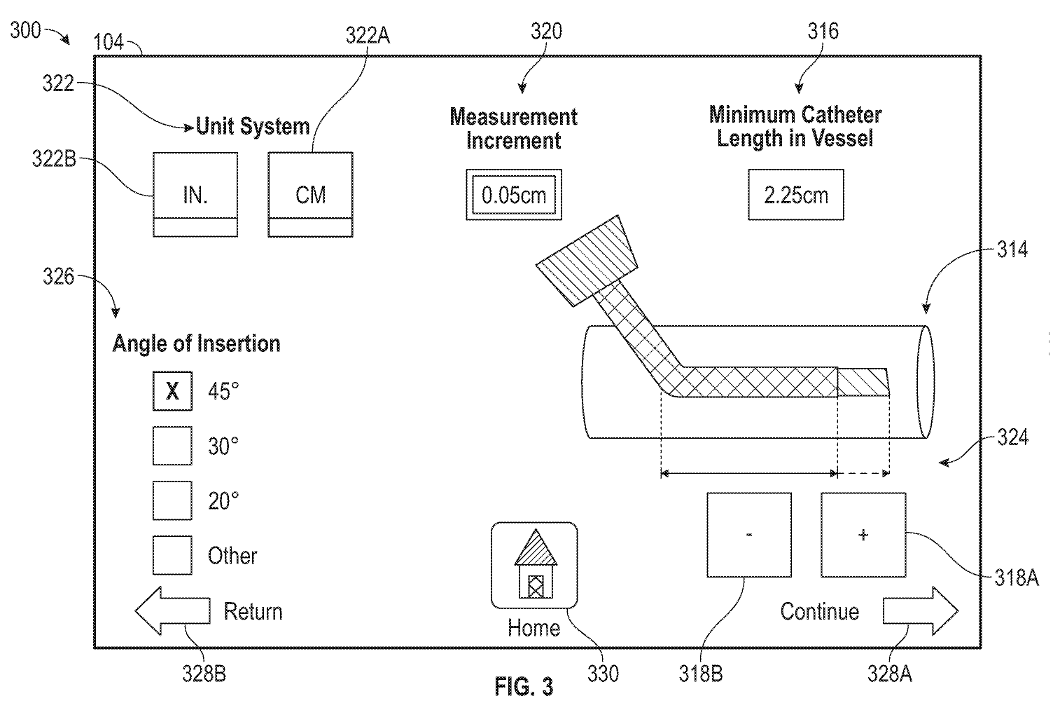

In an embodiment, as shown in FIG. 3, the system 100 can include a parameter input logic 166 configured to provide a parameter input screen 300. The parameter input screen 300 can include a plurality of icons configured to allow a user to modify one or more of the insertion parameters 214. For example, a target vessel icon 314 and/or an indicator icon 316, can correspond to the minimum dwell length parameter 214B. The indicator icon 316 may be configured to be changed to match the minimum dwell length parameter 214B. In an embodiment, the minimum dwell length parameter 214B can be selected by the clinician and entered to the system 100 by way of the parameter input screen 300. In an embodiment, the minimum dwell length parameter 214B can be predetermined by the system 100.

In an embodiment, the clinician can adjust the minimum dwell length parameter 214B. The indicator icon 316 may be configured to be modified incrementally by way of a positive icon 318A or a negative icon 318B. For example, selecting the positive icon 318A can increase the dwell length parameter 214B while selecting the negative icon 318B will decrease the dwell length parameter 214B. The standard unit of measure may be modified by the user by selecting a measurement increment icon 320. The measurement increment icon 320 may be configured to allow the user to change the standard increment of the unit of measure depicted on the display 104. For example, the unit of measure increment may be 0.05 cm. and the user can change the unit of measure increment to be 1.0 cm, 0.1 cm, 0.01 cm. or the like. However, it will be appreciated that greater or lesser increments are also contemplated.

In some embodiments, the parameter input screen 300 includes a unit system icon 322 that allows a user to select between imperial or metric measurement systems of the indicator icon 316. In some embodiments, the user may select the desired units of measure displayed for the indicator icon 316. For example, the indicator icon 316 may be configured to be displayed in metric by selecting the centimeter unit of the unit system icon 322A. However, the user may select the inches unit of the unit system icon 322B which would display the indicator icon 316 in imperial units. In some embodiments, the unit system icon 322 may be configured to change the unit of measurement displayed on all user interfaces on the display 104.

In some embodiments, the icon 314 can further include a catheter dwell length icon 324 indicating, e.g. by an arrow or similar graphical representation, the dwell length of the catheter in the blood vessel. In some embodiments, as the indicator icon 316 is increased or decreased by the positive icon 318A or the negative icon 318B, the catheter dwell length icon 324 may be modified to correspondingly increase or decrease in displayed length.

The parameter input screen 312 may include an angle of insertion (Ɵ) selection panel 326 that includes a selection of predetermined angles of insertion (Ɵ) , for example “45°”, “30°”, “20°” or the like. However, it will be appreciated that other predetermined angles, or predetermined ranges of angles, are also contemplated. In an embodiment, the user can select a specified angle of insertion (Ɵ) by selecting “other” and entering an angle of insertion. Optionally, the angle of insertion (Ɵ) entered by the user may be limited to a predetermined range. As used herein, the angle of insertion (Ɵ) is defined as angle of the catheter relative to a surface of a patient’s skin at the point of insertion. In some embodiments, the “other” option may be configured to allow the user to specify the angle of insertion if the angle is not listed on the angle of insertion selection panel 326.

In some embodiments, the parameter input screen 300 includes one or more arrow icons 328A, 328B to help a user navigate through the user interface of the system 110. In some embodiments, the one or more arrow icons 328A, 328B can be indicated by text that includes “continue” or “return”. In some embodiments, the parameter input screen 300 may include a home icon 330 configured to return the user to a home screen, navigation page, a displayed ultrasound image screen 200, or the like.

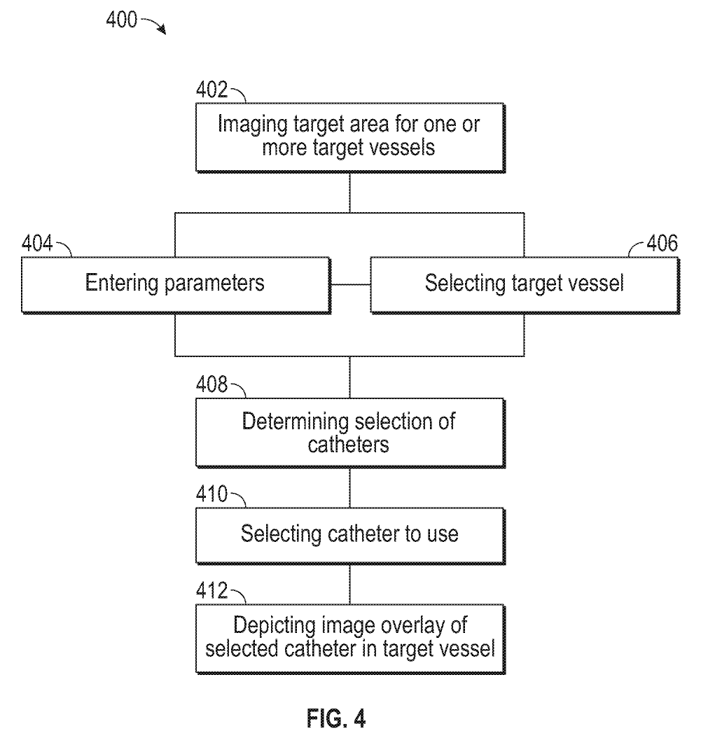

FIG. 4 illustrates a block diagram of a method for selecting a vascular access device for accessing a target blood vessel, in accordance with embodiments disclosed herein. The method 400 includes imaging a target area for one or more blood vessels (block 402). In some embodiments, imaging includes using an ultrasound vessel assessment system 100 having a console 102 including a display 104 and communicatively coupled to an ultrasound probe 106, as described herein.

In some embodiments, the system 100 may be configured to receive a user input to define one or more insertion parameters 214 and determine a selection of VAD’s 80 that may be capable of accessing a target blood vessel 90. The method 400 further includes entering parameters (block 404), such as the angle of insertion, maximum percentage occupancy, and the minimum dwell length as parameters to use for choosing a catheter. In some embodiments, entering includes entering one or more of the angle of insertion, the maximum percentage occupancy, and the minimum catheter dwell length on a parameter input screen 300.

The method 400 can further include selecting one or more target vessels (block 406). The method 400 includes determining a selection of VAD’s 80 (block 408) that are capable of accessing the target vessel 90 based upon one or more insertion parameters 214 such as the angle of insertion, the target vessel depth, the maximum percentage occupancy, and/or the minimum catheter dwell length. The method 400 includes selecting a catheter to use (block 410). In some embodiments, selecting the catheter to use includes selecting from the selection of VAD’s 80 generated by the system 100. The method 400 includes depicting an ultrasound image overlay of the selected catheter in a target vessel 90 (block 412). In some embodiments, the user can move between entering parameters (block 404) and selecting one or more target vessels (block 406) until the user is satisfied with the results.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A vessel assessment system, comprising:
an ultrasound probe; and
a console communicatively coupled to the ultrasound probe, the console comprising a display and one or more logic modules coupled to one or both of a processor and a data store, the one or more logic modules comprising:
an imaging logic configured to provide an ultrasound image from the ultrasound probe on the display, the ultrasound image including one or more blood vessels;
a vessel selection logic configured to superimpose and align one or more vessel selection rings on the ultrasound image; and
a vascular access device (VAD) selection logic configured to automatically select the one or more vessel selection rings, each of the one or more vessel selection rings representing a catheter gauge that can successfully access a blood vessel of the one or more blood vessels on the ultrasound image.

2. The vessel assessment system according to claim 1, wherein the VAD selection logic is configured to retrieve a predetermined insertion parameter and select the one or more vessel selection rings representing one or more catheter gauges that can successfully access the target vessel while remaining within the predetermined insertion parameter.

3. The vessel assessment system according to claim 2, wherein the predetermined insertion parameter includes one or more of a percentage vessel occupancy parameter, a dwell length parameter, and an insertion angle parameter.

4. The vessel assessment system according to claim 2, wherein the predetermined insertion parameter includes a vessel occupancy parameter as a percentage of a diameter of the target vessel.

5. The vessel assessment system according to claim 1, wherein the catheter gauge is the maximum gauge that can access the target vessel.

6. The vessel assessment system according to claim 1, wherein the vessel selection logic is configured to receive an input from a user to manipulate the one or more vessel selection rings.

7. The vessel assessment system according to claim 1, wherein each of the one or more vessel selection rings has a unique color.

8. The vessel assessment system according to claim 1, wherein each of the one or more vessel selection rings is configured to change one or more of a diameter and a shape to align on a blood vessel.

9. The vessel assessment system according to claim 1, wherein each of the one or more vessel selection rings is scaled relative to a blood vessel of the one or more blood vessels to provide a visual comparison of the catheter relative to the blood vessel.

10. The vessel assessment system according to claim 1, wherein the display includes a touch screen, and wherein the vessel assessment system is enabled to receive voice recognition inputs.

11. The vessel assessment system according to claim 1, wherein the imaging logic is configured to provide a subcutaneous depth of the one or more blood vessels on the display.

12. A vessel assessment tool, comprising:
a processor; and
memory communicatively coupled to the processor, the memory comprising machine readable instructions that when executed by the processor, cause the processor to execute:
an imaging logic configured to provide an ultrasound image including one or more blood vessels;
a vessel selection logic configured to superimpose and align one or more vessel selection rings on the ultrasound image; and
a vascular access device (VAD) selection logic configured to automatically select the one or more vessel selection rings, each of the one or more vessel selection rings representing a catheter gauge that can successfully access a blood vessel of the one or more blood vessels on the ultrasound image.

13. The vessel assessment tool according to claim 12, further comprising an ultrasound probe configured to send and receive an acoustic signal, wherein the imaging logic is configured to retrieve information from the ultrasound probe to provide the ultrasound image.

14. The vessel assessment tool according to claim 12, further comprising a console and a display, wherein the display includes a touch screen and is communicatively coupled to a voice recognition input.

15. The vessel assessment tool according to claim 12, wherein the catheter gauge is the maximum gauge that can access the target vessel.

16. The vessel assessment tool according to claim 12, wherein the VAD selection logic is configured to retrieve a predetermined insertion parameter and select the one or more vessel selection rings representing one or more catheter gauges that can successfully access the target vessel while remaining within the predetermined insertion parameter.

17. The vessel assessment tool according to claim 16, wherein the predetermined insertion parameter includes one or more of a percentage vessel occupancy parameter, a dwell length parameter, and an insertion angle parameter.

18. The vessel assessment tool according to claim 16, wherein the predetermined insertion parameter includes a vessel occupancy parameter as a percentage of a diameter of the target vessel.

19. The vessel assessment tool according to claim 12, wherein each of the one or more vessel selection rings has a unique color.

20. The vessel assessment tool according to claim 12, wherein the imaging logic is configured to provide a subcutaneous depth of the one or more blood vessels.
